(19) United States
(12) Patent Application Publication
(10) Pub. No.: US 2022/0007648 A1
(43) Pub. Date: Jan. 13, 2022

(54) Biocidal Compound

(71) Applicant: Xingchen Zhang, Murrysville, PA (US)

(72) Inventor: Xingchen Zhang, Murrysville, PA (US)

(21) Appl. No.: 17/208,092

(22) Filed: Mar. 22, 2021

Related U.S. Application Data
(60) Provisional application No. 63/048,839, filed on Jul. 7, 2020.

(51) Int. Cl. A01N 47/44; A01N 25/30; A01N 25/04

(52) U.S. Cl. CPC A01N 47/44; A01N 25/04; A01N 25/30

(57) ABSTRACT

A polyguanidine biocidal compound miscible with an anionic surfactant. PHMG and PHMB are modified with a variety of different carboxylic acids. Carboxyl groups are added to the ends of the linear polyguanidine chain giving them the structure and properties of an amphoteric surfactant. The modified polyguanidine is now miscible with anionic surfactants while retaining the original bactericidal properties. In this way, it can be compounded with modified polyguanidine and anionic surfactant to produce a product with both sterilization and decontamination properties that is usable in daily use products, and industrial and agricultural fields. A method of manufacturing a polyguanidine biocidal compound is also provided.

### CROSS-REFERENCE TO RELATED APPLICATION

The present application claims priority to, and the benefit of, U.S. Provisional Application No. 63/048,839, which was filed on Jul. 7, 2020 and is incorporated herein by reference in its entirety.

### BACKGROUND

The present invention generally relates to biocidal compounds, and more specifically to an improved polyguanidine fungicide/sterilant/biocidal disinfectant that can be mixed with ionic surfactants without forming precipitation. Accordingly, the present specification makes specific reference thereto. However, it is to be appreciated that aspects of the present invention are also equally amenable to other like applications, devices and methods of manufacture.

Detergents are synthetic, organic, liquid or water-soluble cleaning agents that, unlike soap, are not prepared from fats and oils. They are not inactivated by hard water and have wetting-agent and emulsifying-agent properties. A detergent molecule consists of a long hydrocarbon chain and a water-soluble negative ionic group. An anionic detergent is any of a class of synthetic compounds whose anions are alkali salts, as soap, or whose ions are ammonium salts. Anionic detergent are constructed with a lipophilic hydrocarbon group of the molecule in the form of an anion, or negatively charged ion.

Cosmetics are a category of health and beauty products that are used to care for the face and body or used to accentuate or change a person's appearance. Cosmetics are generally constituted from a mixture of chemical compounds derived from either natural sources or synthetically created ones. Anionic surfactants, the most common of which are the alkyl sulfates, are often the primary ingredient used in cosmetic cleansing products. They are positively charged surfactant ions. Anionic surfactants are those that have a negative charge on their polar head group. They include groups like carboxylic acids, sulfates, sulfonic acids

Surfactants are chemicals that have parts that are both hydrophilic (water loving) and lipophilic (oil loving). This molecular composition means they have the ability to reduce the surface tension when placed into solutions of oil and water. The word “surfactant” is simply a shortened form of the phrase “surface active ingredient.” Anionic surfactants are commonly found in laundry detergents, handwashes, kitchen cleaners, body washes.

Polyhexamethylene guanidine (PHMG) is a guanidine derivative typically used as a biocidal disinfectant. Guanidine is the compound with the formula HNC(NH₂)₂. PHMG in solution has shown to have fungicidal as well as bactericidal activity against both Gram-positive and Gram-negative bacteria. Polyhexamethylene biguanide, (PHMB) is a polymer with antimicrobial and antiviral properties.

Disinfectants are chemical agents or compounds designed to inactivate or destroy microorganisms. A biocide is a chemical substance or microorganism intended to destroy, deter, render harmless, or exert a controlling effect on any undesirable living organism. Fungicides are biocidal chemical compounds or biological organisms used to kill parasitic fungi or their spores.

Currently, powder and liquid detergents, cosmetics, coatings and shampoos with anionic surfactants all cannot be mixed directly with PHMG or PHMB to obtain the disinfecting, biocidal, and fungicidal properties of these compounds. Generally, coagulation occurs when the aqueous solution of cationic surfactant and anionic surfactant is mixed. This happens because the electrostatic attraction between the anion and cation causes the two surfactants to aggregate, and water solubility is greatly reduced, so a precipitation is formed. PHMG and PHMB are polymeric cation surfactants. They are mixed with anionic surfactants to cause coagulation. Daily use cleaning chemicals, such as washing powders, cosmetics, shampoos, body cleaners, and the like, often contain anionic surfactants. Therefore, when PHMG or PHMB are mixed with them, coagulation occurs, and the cleaning and sterilizing effects are lost. As such, these detergents and cosmetics will not have these improved properties.

Therefore, there exists a long felt need in the art for improved detergent and cosmetic cleaning compounds that incorporate the disinfecting, biocidal, and fungicidal properties of PHMG and PHMB. In this manner, the improved fungicidal compounds of the present invention accomplishes all of the forgoing objectives, thereby providing an easy solution enhancing existing detergent and cosmetic cleaning products. A primary feature of the present invention is the use of PHMG and PHMB in detergent and cosmetic cleaning products. Finally, the improved of the present invention is capable of providing an improved polyguanidine fungicide that can be mixed with anionic surfactants without forming precipitation.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The subject matter disclosed and claimed herein, in one embodiment thereof, comprises a polyguanidine biocidal compound. The polyguanidine biocidal compound comprises a polyhexamethylene guanidine (PHMG) molecule. PHMG may be generally defined as (C₇H₁₅N₃)ₙ, wherein n=10-16. The polyguanidine biocidal compound further comprises at least two carboxyl group. The at least two carboxyl groups are grafted to opposing ends of the PHMG molecule. The new polyguanidine biocidal compounds are miscible with anionic surfactants and behave as amphoteric surfactants.

The PHMG molecule may be PHMG hydrochloride, PHMG phosphate, PHMG sulfate, PHMG nitrate, or PHMG an organic acid salt. The organic acid salt is propionate, stearate, or citrate. The carboxyl groups are grafted from a group of polyacids. The polyacids may be tricarboxylic acids, polycarboxylic acids, halogenated acids, halogenated anhydrides, or amino acids.

The subject matter disclosed and claimed herein, in another embodiment thereof, comprises a polyguanidine biocidal compound. The polyguanidine biocidal compound comprises a polyhexamethylene biguanidine (PHMB) molecule. PHMB may be defined generally as (C₈H₁₇N₅)ₙ, wherein n=10-16. The polyguanidine biocidal compound further comprises at least one carboxyl group. The at least one carboxyl groups are grafted to opposing ends of the PHMG molecule. The polyguanidine biocidal compounds are miscible with anionic surfactants and behave as amphoteric surfactants.

The PHMB molecule may be PHMB hydrochloride, PHMB phosphate, PHMB sulfate, PHMB nitrate, or PHMB an organic acid salt. The organic acid salt is propionate, stearate, or citrate. The carboxyl groups are grafted from a group of polyacids. The polyacids may be tricarboxylic acids, polycarboxylic acids, halogenated acids, halogenated anhydrides, or amino acids.

The subject matter disclosed and claimed herein, in another embodiment thereof, comprises a method of manufacturing improved polyguanidine biocidal compounds. The method begins by mixing a polyacid solution and a polyguanidine derivative at a molar ratio of 2:1. The polyacids may be tricarboxylic acids, polycarboxylic acids, halogenated acids, halogenated anhydrides, or amino acids. The polyguanidine derivative is PHMG or PHMB. If the polyacid solution is a solution of amino acids, then the solution of amino acids is prepared into a solution of amino acid ethyl esters. Next, the mixture is heated to between 120 and 180 degrees Celsius. Then, the mixture is stirred at a stir speed between 60 and 120 revolutions per minute, and reacts for approximately four to eight hours. A reactant catalyst is then added. If the polyacid solution was a solution of polycarboxylic acids or halogenated acids, the mixture is neutralized after the chemical reaction is complete to generate a corresponding carboxylate.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They do not intend as an exhaustive description of the invention or do not limit the scope of the invention. Additionally, an illustrated embodiment need not have all the aspects or advantages shown. Thus, in other embodiments, any of the features described herein from different embodiments may be combined.

The present invention provides improved polyguanidine biocides that can be mixed with anionic surfactants without forming precipitation, and changes PHMG and PHMB by grafting polyacids. The improved compounds include properties of zwitterionic surfactants. A zwitterionic surfactant is a surfactant with functional groups, of which at least one has a positive and one has a negative electrical charge. The net charge of the entire molecule is zero. The improved compounds have both decontaminating and sterilizing effects.

The anionic modification of polyguanidine is used to synthesize an improved polyguanidine biocide. The polyguanidine biocide is comprised of a polyguanidine compound that is capable of being mixed with anionic surfactants (ex. detergent) without forming precipitation. This compound provides an alternative to existing polyguanidine compounds that form precipitation.

Ternary and more than ternary polycarboxylic acid, or halogen-polyacid, or amino polyacid is used to chemically graft with PHMG or PHMB to form modified PHMG and PHMB with polyacid groups. The polybasic acid is neutralized with alkali to form its corresponding salt. This allows the PHMG or PHMB have the properties of a part of zwitterionic surfactants allowing them to be completely mixed with anionic surfactants without aggregation. Poly acids used may include citric acid, isocitric acid, high citric acid, high isocitric acid, aconitic acid, homoaconitic acid, trimesic acid, mellitic acid, propane tricarboxylic acid, and ethylenediaminetetraacetic acid. Halogen-polyacids used may include tetrachlorophthalic acid, chlorophthalic acid, bromophthalic acid, and iodophthalic acid. Amino used may include aminomalonic acid, aminosuccinic acid, aminoglutaric acid, aminophthalic acid, diaminophthalic acid.

The present invention, in one exemplary embodiment, are polyhexamethylene guanidine compounds and polyhexamethylene biguanidine compounds. The PHMG and PHMB compounds comprise hydrochloride, phosphate, sulfate, nitrate, and organic salt (propionate, stearate, and citrate). Although the properties of the various salts differ, their bactericidal, antibacterial, and fungicidal properties are basically the same.

PHMG (C₇H₁₅N₃)ₙ may be defined generally as the following chemical formula, or a salt of the same, wherein n=10-16.

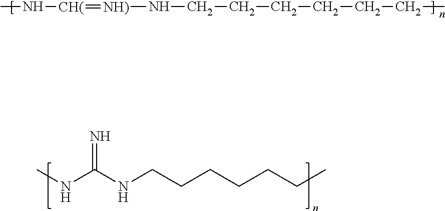

PHMB (C₈H₁₇N₅)ₙ may be defined generally as the following chemical formula, or a salt of the same, wherein n=10-16.

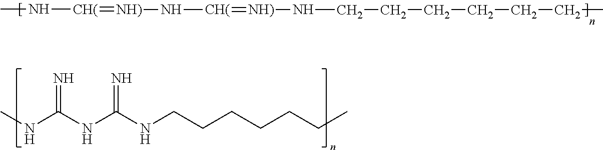

The polyguanidine biocidal compound comprises a polyhexamethylene guanidine (PHMG) molecule or a polyhexamethylene biguanidine (PHMB) molecule. The polyguanidine biocidal compound further comprises at least two carboxyl groups. The carboxyl groups are grafted to opposing ends of the PHMG or PHMB molecule. The polyguanidine biocidal compound is miscible with an anionic surfactant and behaves as an amphoteric surfactant.

The PHMG or PHMB molecule may be PHMG or PHMB hydrochloride, PHMG or PHMB phosphate, PHMG or PHMB sulfate, PHMG or PHMB nitrate, or PHMG or PHMB an organic acid salt. The organic acid salt is propionate, stearate, or citrate. The carboxyl groups are grafted from a group of polyacids. The polyacids may be tricarboxylic acids, polycarboxylic acids, halogenated acids, halogenated anhydrides, or amino acids.

The improved polyguanidine biocide is configured to mix with anionic surfactants without forming precipitation. After PHMG and PHMB are grafted with polycarboxylic acids, there are at least two carboxyl groups on the molecular chain. The carboxyl groups are now ionized into anions. As such, the modified polyguanidine has part of a negative charge. When the modified polyguanidine encounters an anionic surfactant, the negatively charged anions produces an electrostatic repulsion which inhibits precipitation.

PHMG and PHMB are modified with a variety of different carboxylic acids. Carboxyl groups are added to the ends of the linear polyguanidine chain giving them the structure and properties of an amphoteric surfactant. The modified polyguanidine is now miscible with anionic surfactants while retaining the original bactericidal properties. In this way, it can be compounded with modified polyguanidine and anionic surfactant to produce a product with both sterilization and decontamination properties that is usable in daily use products, and industrial and agricultural fields.

PHMG and PHMB both have amino end groups and the guanidino end groups have NH₂ groups. NH₂ groups can undergo condensation polymerization with carboxyl, amine, and halogen groups at high temperatures. Therefore, both ends of the PHMG molecule can be modified with carboxylic acid (anhydride), halogenated carboxylic acid, or amino acids so that the modified PHMG molecule has multiple carboxyl groups at both ends providing the properties of an amphoteric surfactant.

The terminal amine group of PHMG reacts with the carboxyl group of carboxylic acid to remove water molecules and form amine bonds. The terminal amine group of PHMB reacts with the carboxyl group of carboxylic acid to remove water molecules and form amine bonds. The terminal amine group of PHMG reacts with the halogen atom of the halogenated carboxylic acid to remove the hydrogen halide and form a carbon-nitrogen bond (C—N). The terminal amine group of PHMB reacts with the halogen atom of the halogenated carboxylic acid to remove the hydrogen halide and form a carbon nitrogen bond (C—N). The terminal amine group of PHMG reacts with the amino group of the amino acid to remove the ammonia molecule (NH₃) to form a carbon-nitrogen bond. The terminal amine group of PHMB reacts with the amino group of the amino acid to remove the ammonia molecule (NH₃) to form a carbon-nitrogen bond.

The molar ratio of polycarboxylic acid, halogenated polycarboxylic acid, or amino acid to PHMG or PHMB is always 2:1. This is because polycarboxylic acid, halogenated polycarboxylic acid, or amino acid can only react chemically with the two ends of PHMG and PHMB. The direct condensation reaction of carboxylic acid and amine is an important method for synthesizing amides. As long as the water produced in the reaction is removed, the chemical equilibrium will shift to the direction of the product. The carboxyl group of the carboxylic acid undergoes a condensation reaction with the terminal amine of PHMG or PHMB at a temperature above 120 degrees Celsius to form an amide bond which binds the polycarboxyl group to the two ends of the PHMG or PHMB so that each end of the molecular chain of the PHMG or PHMB gains a carboxyl. The chemical reaction of polybasic acid anhydride with PHMG or PHMB also forms amine (amide) bonds, and the chemical reaction is easier to proceed than polybasic carboxylic acid.

Halogens in halogenated hydrocarbons are easily substituted by H₂NR to generate corresponding amine compounds. Alkyl iodide is the most prone to the substitution reaction, followed by bromoalkene, and chloroalkane. The strong carbon-halogen bond of aryl halide makes it more difficult to undergo a similar reaction. Alkaline substances, such as hydroxide sodium, potassium hydroxide, sodium carbonate, potassium carbonate, and potassium iodide may be added to facilitate the reaction. The chemical reaction between the halogen atom of the halogenated polyacid and the amine groups both ends of the PHMG or PHMB removes the hydrogen halide, and the halogenated polyacid is linked to the two ends of the PHMG or PHMB so that there are carboxylic groups added to the molecular PHMG or PHMB chain.

The amino acid must be changed to an amino acid ester prior to the reaction. Each carboxyl group first forms a protective ester, is reacted with the PHMG or PHMB, and is connected to the two ends of the PHMG or PHMB. Finally, hydrolyze the ester bond with the alkali aqueous solution to corresponding sodium salt.

The modified PHMG and PHMB compositions retain the original main polyguanidine structure, with a few carboxyl groups added to both ends or the linear molecule. PHMG and PHMB are sterilized by their polymerized guanidine groups, so the modified compound retains the bactericidal properties. The modification changes the compatibility with anionic surfactants. The new molecules combine the bactericidal properties of the polyguanidine with the decontaminating properties of the anionic surfactants. Without the modification, polyguanidines cannot be effectively mixed with anionic surfactants as coagulation will occur, so they cannot be used simultaneously.

Once the PHMG or PHMB is grafted with the polyacid groups, the polybasic acid is neutralized with an alkali to form its corresponding salt. The polybasic acid refers to an acid containing two or more carboxyl groups. Amino acids contain at least two carboxyl groups and one amino group. Halogenated acids contain at least two carboxyl groups and halogen groups. Polyacids contain at least three carboxyl groups. After grafting the polybasic acid with polyguanidine, it is neutralized to neutrality with alkaline compounds such as sodium hydroxide, sodium carbonate, or ammonia to form its corresponding salt.

At this point, the modified PHMG and PHMB have zwitterionic surfactant properties. Zwitterionic surfactant molecules have cationic and anionic groups. The nature of zwitterionic surfactants is that they can be mixed with cationic and anionic surfactants without precipitation. The modified PHMG and PHMB now both comprise cationic and anionic groups permitting their aqueous solutions to mix with anionic solutions without precipitation.

PHMG and PHMB have hydrochloride, phosphate, sulfate, nitrate, or organic acid salt (propionate, stearate, or citrate) additions. While each salt has different properties, the bactericidal and antibacterial properties are essentially the same. The following chemical reactions use hydrochloride as an example. When the molar ratio of hexamethylene diamine to guanidine hydrochloride is 1:1, the molecular structure is H[HN—(CH₂)₆—NH—C(═NH.HCl)]ₙ—H, wherein n=10-16. When the molar ratio of hexamethylene diamine to guanidine hydrochloride is greater than 1:1, the molecular structure is H—[HN—(CH₂)₆—NH—C(═NH.HCl)]n-NH₂, wherein n=10-16.

The PHMG polymer has two amine groups located at the ends. One amino group and one guanidine group as terminal groups, and two guanidine groups as terminal groups. When the molar ratio of hexamethylene diamine to guanidine hydrochloride is 1:1, one amino group and one guanidine group are the main end groups. When the molar ratio of hexamethylene diamine to guanidine hydrochloride is greater than 1:1, the two end groups are mainly amino groups. Both amino groups and guanidino end groups have NH₂ groups. NH₂ groups can undergo condensation polymerization with carboxyl, amine, and halogen groups at high temperatures. Therefore, both ends of the PHMG can be modified with carboxylic acid (anhydride), halogenated carboxylic acid, and amino acid so that the modified PHMG molecule has multiple carboxylic groups at both ends, and has the properties or an amphoteric surfactant.

The situation with PHMB are the same as for PHMG. PHMB can also be modified similarly with carboxylic acid (anhydride), halogenated carboxylic acid, and amino acid. The modified PHMB molecule has multiple carboxylic groups at both ends and has the properties or an amphoteric surfactant.

Tables 1-3 provide a list of acceptable polyacid ingredients to react with the PHMG and PHMB.

TABLE 1
Tricarboxylic acids and polycarboxylic acids

| Name | Chemical Name | Formula |
|---|---|---|
| Citric Acid | 2-hydroxypropane-1,2,3-tricarboxylic | C6H8O7 |
| | acid | |
| Isocitrate | 1-hydroxypropane-1,2,3-tricarboxylic | C₆H₈O₇ |
| | acid | |
| Aconitic acid | 1-propylene-1,2,3-tricarboxylic acid | C₆H₆O₆ |
| Propane-1,2,3-tricarboxylic acid | Propane-1,2,3-tricarboxylic acid | C₆H₈O₆ |
| High citric acid | 2-hydroxybutane-1,2,4-tricarboxylic | C₇H₁₀O₇ |
| | acid | |
| High isocitrate | 1-hydroxybutane-1,2,4-tricarboxylic | C₇H₁₀O₇ |
| | acid | |
| High aconitic acid | Prop-1-ene-1,2,3-tricarboxylic acid | C₇H₈O₆ |
| Trimesic acid | Benzene-1,3,5-tricarboxylic acid | C₉H₆O₆ |
| Trimellitic acid | Benzene-1,2,4-tricarboxylic acid | C₉H₆O₆ |
| Graphic acid | Benzene-1,2,3,4,5,6-hexacarboxylic | C₉H₆O₆ |
| Benxenehexacarboxylic acid | acid | |

TABLE 2
Halogenated acids and halogenated anhydrides

| Name | Chemical Name | Formula |
|---|---|---|
| Tetrachlorophthalicacid | 3,4,5,6- tetrachloro-2-benzenedicarboxylicacid; | C8H2Cl4O4 |
| | 3,4,5,6- tetrachloro-1,2-benzenedicarboxylicacid | |
| Tetrachloroterephthalic acid | 2,3,5,6- tetrachlorobenzene-1,4-dicarboxylate; | C8Cl4O4H2 |
| | 3,4,5,6-Tetrachlorophthalic Acid | |
| 2,4,5,6-tetrachloroisophthalic | 2,4,5,6-tetrachloroisophthalic acid | C8H2Cl4O4 |
| acid | | |
| 4-chloroisophthalicacid | 4-Chlorobenzene-1,3-dicarboxylic acid; | C8H5ClO4 |
| | 4-chloroisophthalic acid | |
| 5-Chloroisophthalic Acid | 5-Chloroisophthalic Acid | C₈H₅ClO₄ |
| 4-Chloroisophthalic Acid | 4-Chloroisophthalic Acid | C8H5ClO4 |
| Tetrachlorophthalic anhydride | Tetrachloro-1,2-benzenedicarboxylicacidanhydride | C8Cl4O3 |
| 4-Chlorophthalic anhydrid | 4-Chlorophthalic anhydrid | C8H3ClO3 |
| 3-chlorophthalicacid | 3-Chloro-1,2-benzenedicarboxylicacid | C8H5ClO4 |
| 4,5-Dichlorophthalic acid | 4,5-Dichlorophthalic acid | C8H4Cl2O4 |
| 2,5-Dichloroterephthalic acid | 2,5-Dichloroterephthalic acid | C8H4Cl2O4 |
| 4,5-Dichlorophthalic acid | 4,5-Dichloro-2-carboxybenzoi | C8H4Cl2O4 |
| 4,5-dichlorophthalic | 4,5-dichlorophthalic anhydride | C8H2Cl2O3 |
| anhydride | | |
| Phthalic anhydride | 3,6-Dichlorophthalic anhydride; | C8H2Cl2O3 |
| | 3,6-Dichlorophthalic acid anhydride | |
| Tetrafluorophthalic | 3,4,5,6-Tetrafluorophthalic anhydride | C8F4O3 |
| Anhydride | | |
| 5-Fluoro-isobenzofurandione | 3-Fluorophthalic Anhydride | C8H3FO3 |
| 4-Fluorophthalic anhydride | 4-Fluorophthalic anhydride | C8H3FO3 |
| 4,5-Difluorophthalic | 4,5-Difluorophthalic Anhydride | C8H2F2O3 |
| Anhydride | | |
| Tetrafluorophthalic Acid | Tetrafluorophthalic Acid | C8H2F4O4 |
| 4-Fluorophthalic acid | 4-Fluorobenzene-1,2-dicarboxylic acid | C8H5FO4 |
| 3-Fluorophthalicacid | 3-Fluorophthalicacid | C8H5FO4 |
| 4,5-Difluorophthalic acid | 4,5-Difluorobenzene-1,2-dicarboxylic acid | C8H4F2O4 |
| 3,6-Difluorophthalic acid | 3,6-Difluorophthalic acid | C8H4F2O4 |
| 3-Bromophthalic acid | 3-Bromophthalic acid | C8H5BrO4 |
| 4-Bromophthalic acid | 4-Bromo-1,2-benzenedicarboxylic acid | C8H5BrO4 |
| 5-Bromoisophthalic acid | 5-Bromoisophthalic acid | C8H5BrO4 |
| 4-Bromoisophthalic acid | 1,3-Dicarboxy-4-bromobenzene; | C8H5BrO4 |
| | 1-Bromobenzene-2,4-dicarboxylic acid; | |
| | 4-Bromo-1,3-benzenedicarboxylic acid | |
| 2,5-Dibromoterephthalic acid | 1,4-Dibromo-2,5-benzenedicarboxylic acid; | C8H4Br2O4 |
| | 2,5-Dibromo-4-carboxybenzoic acid; | |
| | 2,5-Dibromoterephthalic acid | |
| Tetrabromoterephthalic acid | 2,3,5,6-Tetrabromoterephthalicacid | C8H2Br4O4 |
| Tetrabromophthalic | 4,5,6,7-four bromine phthalate [acid] anhydride | C8Br4O3 |
| anhydride | | |
| 4-Bromophthalic anhydride | 4-Bromophthalic anhydride; | C₈H₃BrO₃ |
| | 5-Bromo-1,3-isobenzofurandione | |
| 3-bromophthalic anhydride | 3-bromophthalic anhydride | C8H3BrO3 |
| 4-iodophthalic acid | 4-iodophthalic acid | C8H5IO4 |
| 2-iodoterephthalic acid | 2-Iodo-1,4-benzenedicarboxylicacid | C8H5IO4 |
| 4,5-diiodophthalic acid | 4,5-diiodophthalic acid | C8H4I2O4 |
| 4,5,6,7- | 4,5,6,7-tetraiodo-2-benzofuran-1,3-dione; | C8I4O3 |
| Tetraiodoisobenzofuran-1,3- | 1,3-Isobenzofurandione, 4,5,6,7-tetraiodo | |
| dione | | |
| 3-iodophthalic anhydride | 3-iodophthalic anhydride | C8H3IO3 |
| 2-Chloroglutaric acid | Pentanedioic acid, 2-chloro | C5H7ClO4 |
| (S)-2-chlorosuccinic acid | (S)-2-chlorosuccinic acid | C4H5ClO4 |
| (S)-2-Bromoglutaric cid | (S)-2-Bromoglutaric Acid | C5H7BrO4 |
| Bromosuccinic acid | 2-Bromobutanedioicacid | C4H5BrO5 |

TABLE 3
Amino Acids

| Name | Chemical Name | Formula |
|---|---|---|
| L-Glutamic acid | Aminoglutaric acid | C5H9NO4 |
| Aminomalonic acid | Aminomalonic acid; 2-aminopropanedioate | C3H5NO4 |
| Aspartic acid | 2-Aminosuccinic acid | C4H7NO4 |
| 4-Aminophthalic acid | 4-Aminophthalic acid | C8H7NO4 |
| 5-Aminoisophthalic acid | 5-Aminoisophthalic acid | C8H7NO4 |
| 2,5-Diaminoterephthalic acid | 2,5-Diaminoterephthalic acid | C8H8N2O4 |
| Cyclohexylene | Cyclohexylene Diaminetetracetic Acid | C14H19N2O8 |
| Diaminetetracetic acid | | |
| 2,3-Diaminobutyric acid | 2,3-Diaminobutyric acid | C4H10N2O2 |
| 2-Aminoterephthalic acid | 2-Aminoterephthalic Acid | C8H7NO4 |
| 3-Aminophthalic acid | 3-Aminophthalic acid | C8H7NO4 |
| Cyclohexanediaminetetraacetic | 1,2-cyclohexanediamine-N,N,N′,N′-tetraacetic acid; | C14H22N2O8 |
| acid | 1,2-CYCLOHEXANEDIAMINETETRAACETIC ACID; | |
| | 1,2-CYCLOHEXYLENEDIAMINETETRAACETIC ACID | |
| Ethylenediaminetetraacetic | Ethylenediaminetetraacetic acid□ EDTA) | C10H16N2O8 |
| acid□ EDTA) | | |

For convenience and to illustrate that its two end groups are NH₂ groups, the PHMG and PMHB molecules are written as NH₂-PHMG-NH₂ and NH₂-PHMG-NH₂ respectively for the following examples.

### Example 1

PHMG or PHMB reacting with a polycarboxylic acid (COOH group >3 and =3) from Table 1. Using the example of citric acid, the following reactions occur:

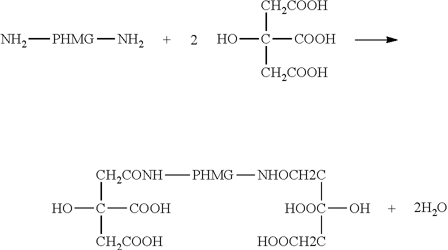

The terminal amide group of PHMG reacts with the carboxyl group of carboxylic acid to remove the water molecules and form the amide bonds.

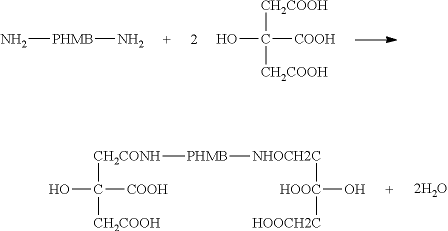

The terminal amide group of PHMB (PHMG) reacts with the carboxyl group of carboxylic acid to remove the water molecules and form the amide bonds.

### Example 2

PHMG or PHMB reacting with a halogenated diacid from Table 2. Using the example of halogenated malonic acid, the following reactions occur:

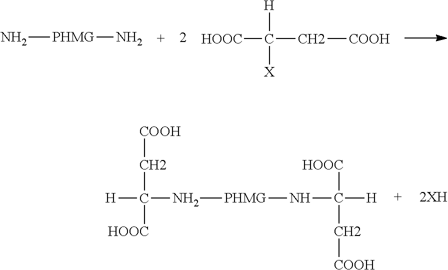

### Where X═F, Cl, Br, or I

The terminal amide group of PHMG reacts with the halogen atom of the halogenated carboxylic acid to remove the hydrogen halide and form a carbon-nitrogen bond (C—N).

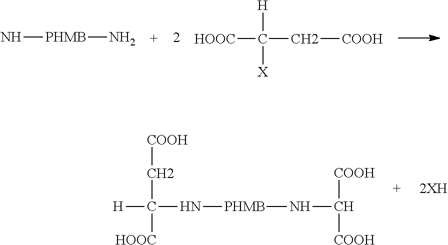

### Where X═F, Cl, Br, or I

The terminal amide group of PHMB reacts with the halogen atom of the halogenated carboxylic acid to remove the hydrogen halide and form a carbon-nitrogen bond (C—N).

### Example 3

PHMG or PHMB reacting with an amino diacid from Table 3. Using the example of aminomalonic acid, the following reactions occur:

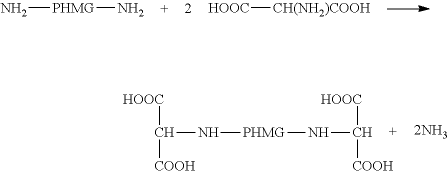

The terminal amide group of PHMG reacts with the amino group of the amino acid to remove the ammonia molecule NH₃ to form a carbon-nitrogen bond (C—N).

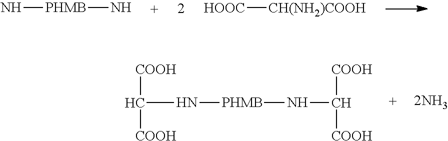

The terminal amide group of PHMB(PHMG) reacts with the amino group of the amino acid to remove the ammonia molecule NH₃ to form a carbon-nitrogen bond (C—N).

A method of manufacturing a polyguanidine biocidal compound is also provided. The method begins by mixing a polyacid solution and a polyguanidine derivative solid or water solution at a molar ratio or 2:1 in a flask or conventional airtight reaction kettle. The polyacids used in the solution may be tricarboxylic acids, polycarboxylic acids, halogenated acids, halogenated anhydrides, or amino acids as illustrated in Tables 1-3. The polyguanidine derivative is PHMG or PHMB. Next, the mixture is heated to between approximately 120 and 180 degrees Celsius. The reaction temperature of polycarboxylic acid or polybasic acid and PHMG or PHMB is between 120 and 180 degrees Celsius with a reaction time of approximately between four to eight hours.

Then, the mixture is stirred at a stir speed between 60 and 120 revolutions per minute. A reactant catalyst is then added. The catalyst is added to the mixture at temperature below 100 degrees Celsius. Add one to two percent of the total reactant catalyst, basic catalyst: sodium hydroxide, potassium hydroxide, sodium carbonate, or potassium carbonate. Catalyst: potassium iodide. Potassium iodide has a better catalytic effect, because potassium iodide can convert fluorine, chlorine, and bromine halides into iodine, and iodide is easily removed.

If the polyacid solution is a solution of amino acids, then the solution of amino acids is prepared into a solution of amino acid ethyl esters. This is necessary to protect each carboxyl group. The reaction temperature is still between 120 and 180 degrees Celsius. After the chemical reaction is complete, the temperature is reduced to approximately 70 degrees Celsius, and the ester bond is hydrolyzed into the corresponding sodium salt with an alkaline aqueous solution (sodium hydroxide solution or sodium carbonate solution).

All chemical modifications to the PHMG or PHMB may be solid PHMG or PHMB or liquid PHMG or PHMB solutions with different contents. PHMG or PHMB is added first, followed by the aqueous solution of the modifier. The room temperature is then raised to approximately 100 degrees Celsius, the water is evaporated, and the temperature is then raised to 120 degrees Celsius to start the chemical reaction.

The PHMG or PHMB and polycarboxylate acid reaction is a chemical reaction of dehydration to generate amide bonds. The reaction with halogenated polyacids is a chemical reaction that removes hydrogen halides to form carbon-nitrogen bonds. The reaction with amino acids is a reaction that removes ammonia to form carbon-nitrogen bonds. The chemical reaction continues to react between 120 to 180 degrees Celsius and needs to be slowly raised. The chemical reaction ends at 180 degrees Celsius.

The hydrogen halide, ammonia, and other gasses released during the reaction are removed to avoid environmental pollution. After the reaction, the temperature is slowly lowered and the solid product is cooled to approximately 100 degrees Celsius and discharged, then cooled and crushed. When the liquid product is cooled to approximately 90 degrees Celsius, pure water is added to the flask or reaction kettle, diluted to the desired concentration, and discharged.

If the polyacid solution was a solution of polycarboxylic acids or halogenated acids, the mixture is neutralized after the chemical reaction is complete to generate a corresponding carboxylate. After the chemical reaction is complete, modifications of polycarboxylic acids or halogenated acids and PHMG or PHMB are neutralized with sodium hydroxide or sodium carbonate. The aqueous solution should have a final pH of approximately 7-8.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A polyguanidine biocidal compound comprising:
a polyhexamethylene guanidine (PHMG) molecule having the following formula: 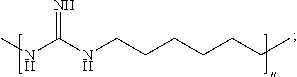
and
at least one carboxyl group grafted to each opposing end of the PHMG molecule; and
wherein n=10-16.

2. The polyguanidine biocidal compound of claim 1, wherein the polyguanidine biocidal compound is miscible with an anionic surfactant.

3. The polyguanidine biocidal compound of claim 1, wherein the PHMG molecule is PHMG hydrochloride, PHMG phosphate, PHMG sulfate, or PHMG nitrate.

4. The polyguanidine biocidal compound of claim 1, wherein the PHMG molecule is a PHMG organic acid salt.

5. The polyguanidine biocidal compound of claim 4, wherein the organic acid salt is propionate, stearate, or citrate.

6. The polyguanidine biocidal compound of claim 1, wherein the carboxyl groups are grafted from a group of tricarboxylic acids or polycarboxylic acids.

7. The polyguanidine biocidal compound of claim 1, wherein the carboxyl groups are grafted from a group of halogenated acids or halogenated anhydrides.

8. The polyguanidine biocidal compound of claim 1, wherein the carboxyl groups are chemically grafted from a group of amino acids.

9. A polyguanidine biocidal compound comprising:
a polyhexamethylene biguanidine (PHMB) molecule having the following formula: 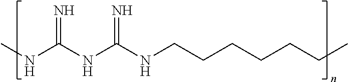
and
at least one carboxyl group grafted to each opposing end of the PHMB molecule; and
wherein n=10-16.

10. The polyguanidine biocidal compound of claim 9, wherein the polyguanidine biocidal compound behaves as an amphoteric surfactant.

11. The polyguanidine biocidal compound of claim 9, wherein the PHMB molecule is PHMB hydrochloride, PHMB phosphate, PHMB sulfate, or PHMB nitrate.

12. The polyguanidine biocidal compound of claim 9, wherein the PHMB (PMGH) molecule is a PHMB organic acid salt.

13. The polyguanidine biocidal compound of claim 13, wherein the organic acid salt is propionate, stearate, or citrate.

14. The polyguanidine biocidal compound of claim 9, wherein the carboxyl groups are grafted from a group of tricarboxylic acids or polycarboxylic acids.

15. The polyguanidine biocidal compound of claim 9, wherein the carboxyl groups are grafted from a group of halogenated acids or halogenated anhydrides.

16. The polyguanidine biocidal compound of claim 9, wherein the carboxyl groups are chemically grafted from a group of amino acids.

17. A method of manufacturing a polyguanidine biocidal compound comprising:
mixing a polyacid solution and a polyguanidine derivative solid or water solution at a molar ratio or 2:1;
heating the mixture to between 120 and 180 degrees Celsius;
stirring the mixture at a stir speed between 60 and 120 revolutions per minute;
adding a reactant catalyst;
reacting the mixture for between approximately four to eight hours; and
neutralizing the product to a pH of 7-8 to generate a corresponding carboxylate.

18. The method of claim 17, wherein the polyacid solution comprises tricarboxylic acids, polycarboxylic acids, halogenated acids, halogenated anhydrides, or amino acids.

19. The method of claim 17, wherein the polyguanidine derivative is PHMG or PHMB.

20. The method of claim 17, wherein if the polyacid solution is a solution of amino acids, first preparing the solution of amino acids into a solution of amino acid ethyl esters.
